(19) United States
(12) Patent Application Publication
(10) Pub. No.: US 2014/0058445 A1
(43) Pub. Date: Feb. 27, 2014

(54) HIGH TENSION SUTURE ANCHOR

(71) Applicant: Terry Mattchen, Scottsdale, AZ (US)

(72) Inventor: Terry Mattchen, Scottsdale, AZ (US)

(21) Appl. No.: 13/783,061

(22) Filed: Mar. 1, 2013

Related U.S. Application Data
(60) Provisional application No. 61/741,792, filed on May 14, 2012.

(51) Int. Cl. A61B 17/04

(52) U.S. Cl. CPC A61B 17/0401
USPC 606/232

(57) ABSTRACT

The present invention describes a bio-medically compatible gripping device capable of radial collapse in accordance with the shrinkage of a nylon or other polymeric material cored surgical cable undergoing tension while maintaining a firm grip throughout the process. It provides a gripping device capable of maintaining a grip on the outer surface of a slippery delicate cable, the grip being approximately uniform along both the length and circumference of the cable. The present invention also provides a gripping device capable of maintaining a grip, yet not damage, a delicate cable under high tension for a period of time adequate for as the healing process to occur.

### BACKGROUND OF THE INVENTION

1. Field of the Invention

This invention relates to devices for retaining surgical cables under high tension.

2. Description of the Related Art

Present day polymer based surgical cables seem to defy all attempts to engage and lock the under high tension, thereby dramatically curtailing their range of practical use. Cable locks such as described in U.S. Pat. No. 7,625,373 attempt to solve the just such a problem. U.S. Pat. No. 7,625,373 is a typical example of the use of a wedge as a simple machine for securement.

Material properties of polymer fibers tend to complicate the situation. Surfaces tend to be slippery and materials have hysteresis. In some circumstances the materials tend to deform and flow (as implied by the common term “plastic”). Conventional knots are inadequate; clasps and fasteners slip under high tension. Attempts to counteract slippage by application of increased pressure often result in cutting or fraying of the cable. Despite their usefulness, widespread acceptance of polymer cables depends in part on the availability of an efficient, economical, convenient, and reliable means of clamping and retaining under moderate to high tension.

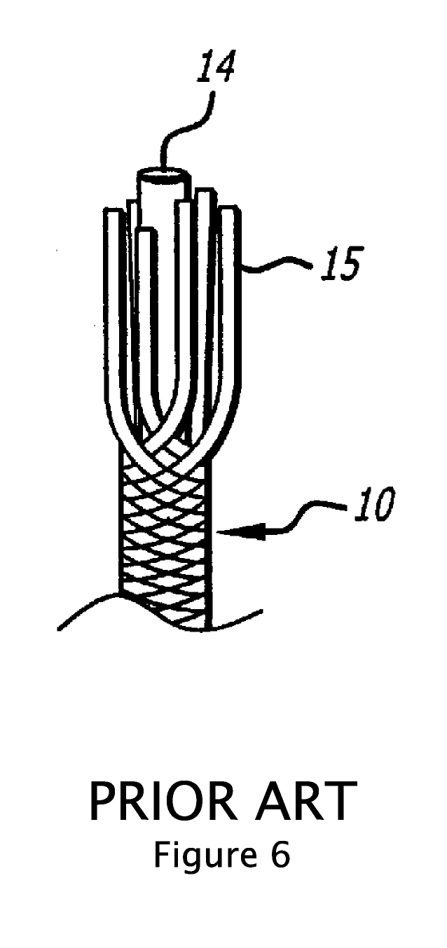

Nevertheless, as disclosed in U.S. Pat. No. 6,589,246, certain polymer cables have shown promise for surgical use. The cable (10) of the '246 patent, shown in FIG. 6, is a composite. The nylon or other polymeric material core (14) comprises about 75% of the diameter of the finished cable; the high strength UHMW polyethylene fibers (15) braided onto the core comprise the remaining 25%.

The difficulties that are unique to gripping such braided polymer cable over the conventional steel cable are the following:
The polymer cable stretches under high tension. As tension is applied, the diameter of the nylon or other polymeric material core shrinks by approximately 12%. Thus, the gripping device must be flexible enough to collapse correspondingly, all the while maintaining a still firmer grip on the cable. By contrast, prior art steel cables do not shrink under tension, thereby obviating the need for a flexibly collapsing gripping mechanism.
The fibers of the braided jacket do not stretch along with the core. Because such fibers are extremely delicate, (i.e., each fiber having a diameter measuring only a few ten-thousandth's of an inch), they will break if not gripped gently and uniformly about their circumference. By contrast, prior art steel cables have a robust metallic surface and are therefore not sensitive to asymmetric gripping.
Finally, the polyethylene fibers of the jacket are quite slippery, and so must be held gently, uniformly, and yet still quite firmly.

In essence, the force F required to retain such a cable must be applied in such a way that the cable does not cause damage, yet be of sufficiently significant magnitude to maintain a grip under high tension as the healing process evolves.

One way to accomplish this goal is to apply the force over a large surface area, i.e.

F=∫Pda

Where P is the applied pressure and the integral is taken over the contacting surface area of the cable. Although the pressure indeed will vary to some degree from point to point, it is desirable to maintain a consistent a value as possible in order to avoid shearing or tearing of the surface fibers. An application of 30 lbs. of force for example, can be achieved by 3000 psi over an area of 0.01 square inches, or by 300 psi over an area of 0.10 square inches. The latter is a more optimal choice given the delicate nature of cables such as described in the '246 patent. Consequently, a device capable of applying a relatively constant pressure P over a relatively large area of the cable surface resulting in an adequate value for the total integrated force, F, is desirable.

### SUMMARY

The above concerns are met by a surgical cartridge comprised of two components, a cartridge and an insertable collet with a star-shaped cross section. The collet of the present design is comprised of several gripping fingers that move radially inward to grip the cable. The fingers close in on the cable in a uniform manner, while maintaining as much contact area as possible. This design is further optimized by the number of fingers, the thickness of the resulting finger wall vs. the size of the cable and surgical cartridge. For example, an acceptable balance was achieved via a nine finger collet design. A seven finger design, although functional, required an unacceptably high crimping. A ten finger design could be functional using a bigger cable; however, the resulting wall thickness of the fingers becomes unworkably thin. Consequently, a delicate balance must be achieved between the number of fingers and the thickness of the finger wall.

More precisely, it is an objective of the present invention to provide a gripping device capable of providing a firm, gentle, and uniform pressure to a surgical cable comprised of a nylon or other polymeric material core covered by a polyethylene fiber braid. It is a further objective of this invention to provide a gripping device that is biomedically compatible with the human body.

It is a still further objective of this invention to provide a gripping device capable of radial collapse in accordance with the shrinkage of a nylon or other polymeric material cored surgical cable undergoing tension while maintaining a firm grip throughout the process.

It is a still further objective of this invention to provide a gripping device capable of maintaining a grip on the outer surface of a slippery delicate cable, the grip being approximately uniform along both the length and circumference of the cable. It is a still further objective of this invention to provide a gripping device capable of maintaining a grip, yet not damage, a delicate cable under high tension for a period of time adequate for the healing process to occur.

A bio-compatible high tension suture anchor capable of providing a consistently uniform pressure of 2500-3500 psi over an area measuring less than 0.05 square inches is disclosed. The bio-compatible high tension suture anchor may further comprise a truncated, hollow, conical cylinder having a length, a radius, and a cylindrical wall. The cylindrical wall may be comprised of a regular series of ridges and valleys parallel to the length of the conical cylinder. It may also comprise a retaining collar operable for progressively compressing the radius of the conical cylinder upon insertion of the conical cylinder within the retaining collar. The regular series of ridges and valleys may define a radial arrangement of compressive fingers, the fingers being operable for radially uniform compression of a cable inserted within the hollow space of the conical cylinder. The radially uniform compression may be approximately invariant over the length of the conical cylinder. The radially uniform compression may be consistent over cable diameter shrinkages of up to 15%. The bio-compatible high tension suture anchor may have 7-11 radial fingers. The radial fingers may have a wall thickness of 0.012-0.012 inches. The high tension suture may be made of a metallic alloy. The metallic alloy may be titanium.

The bio-compatible high tension suture anchor may also have a crimping tube integral with the suture anchor and parallel to the length of the conical cylinder. Alternatively, it may include a pair of opposing attachment tabs integral with the suture anchor and perpendicular to the length of the conical cylinder.

An exemplary installation tool for deployment of the high tension suture anchor is also described and claimed. The tool comprises an upper forked member and a lower forked member. The upper forked member and the lower forked member are configured in a scissor-like fashion. The lower forked member is operable for securing and positioning the high tension suture anchor. The tool also comprises an auxiliary lever operable for gripping a surgical cable threaded through the high tension suture anchor and a positioning conduit operable for positioning and tightening a surgical cable to the desired level of tension. The tool further comprises a tension retaining member operable for maintaining the desired level of tension on the surgical cable throughout the deployment process.

A method of deploying a surgical cable within a fractured bone is described and claimed as well. The method comprises the steps of:
1) Presenting any one of the above described high tension suture anchors,
2) Presenting an installation tool as previously described
3) Positioning the anchor within the tool, thereby forming a tooled anchor assembly,
4) Threading a surgical cable through the tooled anchor assembly,
5) Pressing the anchor assembly through a surgically drilled bone aperture while pulling the surgical cable taut,
6) Squeezing the upper and lower forked members of the installation tool together, while urging the conical cylinder of the anchor into the throat of its retaining collar, thereby gripping and securing the inserted surgical cable,
7) Releasing the tension on the surgical cable,
8) Sliding the upper and lower fork members away from the anchor assembly, and
9) Cutting the extraneous length of the surgical cable.

A method of employing cerclage to an assembly of fractured bones using a surgical cable is also described and claimed. The method comprises the steps of:
1) Presenting a high tension suture anchor having an integrated crimping tube,
2) Presenting an installation tool as previously described,
3) Positioning the anchor within the tool, thereby forming a tooled anchor assembly,
4) Crimping one end of the surgical cable in the crimping tube,
5) Encircling the assembly of fractured bones with the free end of the surgical cable,
6) Threading the free end of the surgical cable through the tooled anchor assembly,
7) Squeezing the upper and lower forked members of the installation tool together, while urging the conical cylinder of the anchor into the throat of its retaining collar, thereby gripping and securing the inserted surgical cable,
8) Releasing the tension on the surgical cable,
9) Sliding the upper and lower fork members away from the anchor assembly, and
10) Cutting the extraneous length of the surgical cable.

A method of deploying a surgical cable to the surface of a fractured bone is described and claimed. The method comprises the steps of:
1) Presenting a high tension suture anchor with opposing tabs,
2) Presenting an installation tool as previously described,
3) Positioning the anchor within the tool, thereby forming a tooled anchor assembly,
4) Threading a surgical cable through the tooled anchor assembly,
5) Pressing the anchor assembly against the surface of the fractured bone such that the opposing tabs lie flat against the bone,
6) Screwing the anchor assembly to the fractured bone using the opposing tabs,
7) Pulling the surgical cable taut,
8) Squeezing the upper and lower forked members of the installation tool together, while urging the conical cylinder of the anchor into the throat of its retaining collar, thereby gripping and securing the inserted surgical cable,
9) Releasing the tension on the surgical cable,
10) Sliding the upper and lower fork members away from the anchor assembly, and
11) Cutting the extraneous length of the surgical cable.

These, other features, and various advantages will be apparent to those skilled in the art from the following detailed description of the preferred embodiments and accompanying drawings.

### DETAILED DESCRIPTION OF THE PREFERRED EMBODIMENT

### Description of the Items in the Figures

10—cable (prior art)
14—polymeric material core (prior art)
15—high strength UHMW polyethylene fiber braid (prior art)
101—surgical cartridge
102—bone fragments
103—surgical cable
104—optional washer
105—outer bone aperture for surgical cable (103)
201—retaining collar
202—collet having a star-shaped cross section
203—central void
302—contacting surface of cable (103)
303—inside surface of collet wall
304—outside surface of collet wall
305—collet finger
401—radial cross section of uncompressed collet with cable (103) inserted
402—radial cross section of collet compressed around cable (103)
501—radial plane near top of collet (202)
502—radial plane approximately one third collet length from the top of collet (202)
503—radial plane approximately two thirds collet length from the top of collet (202)
504—radial plane near bottom of collet (202)
700—exemplary installation tool with cable gripping member (703)
701—upper forked member
702—lower forked member
703—cable gripping member
704—tension retaining member
800—installed suture anchor
801—simple embodiment of installation tool
901—cerclage cartridge
1001—tabbed cartridge

### DESCRIPTION OF THE FIGURES

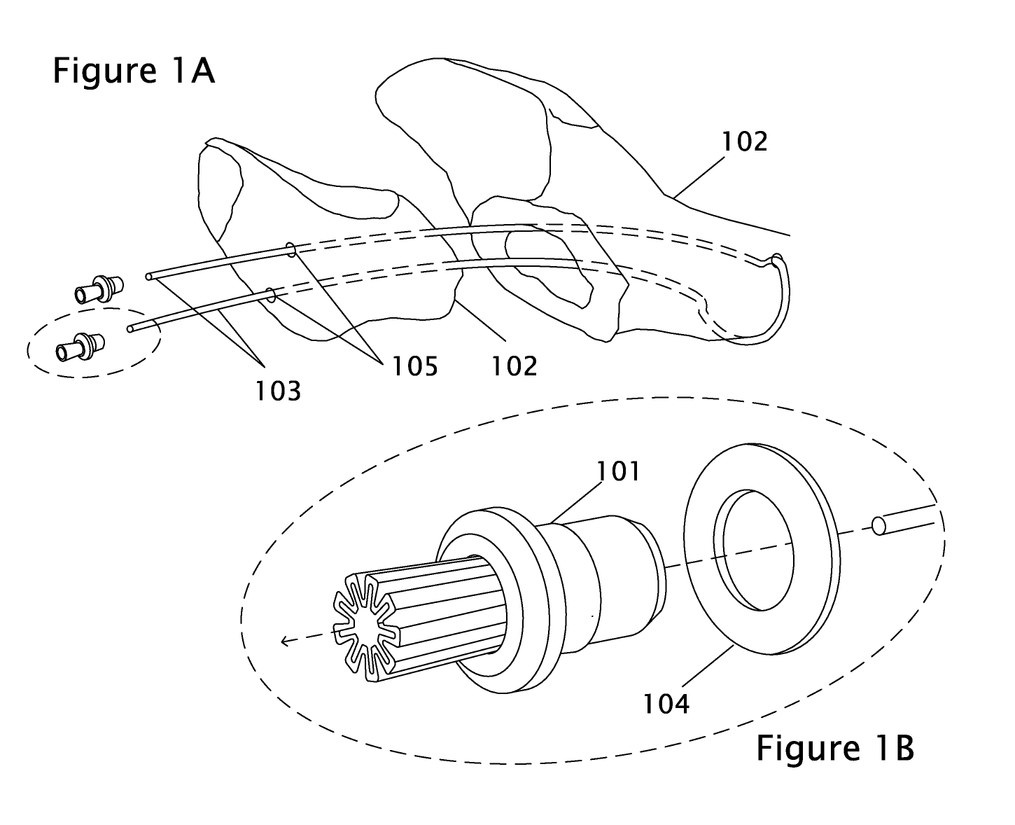

FIG. 1: An illustration of the scenario wherein the surgical cartridge (101) of the present invention is used to secure bone fragments (102). FIG. 1A indicates and overall view. FIG. 1B shows an expanded view of the dotted oval region in FIG. 1A.

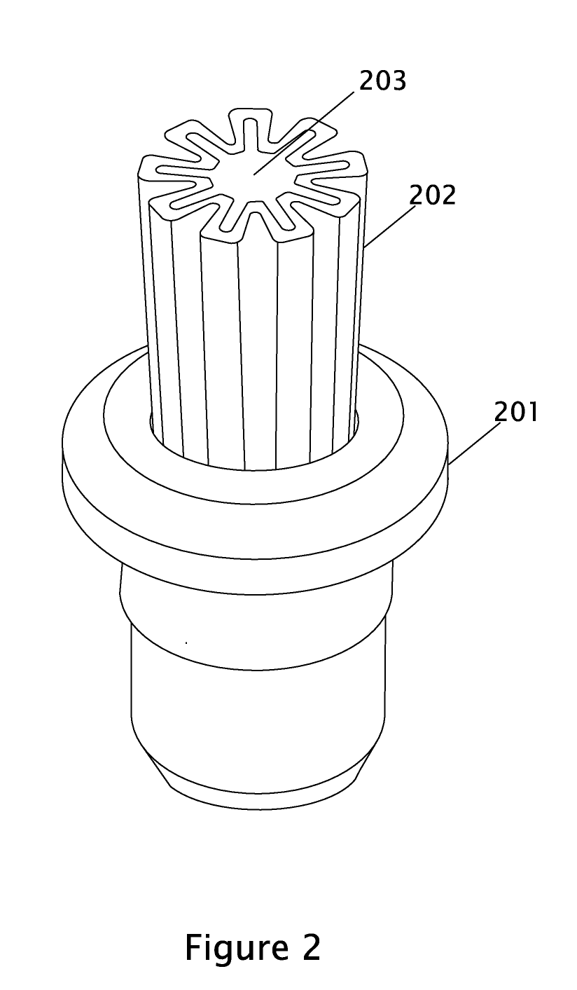

FIG. 2: The surgical cartridge (101) is shown in greater detail. The collet (202) has a star-shaped cross section and surrounds a central void (203) that is enclosed by a retaining collar (201).

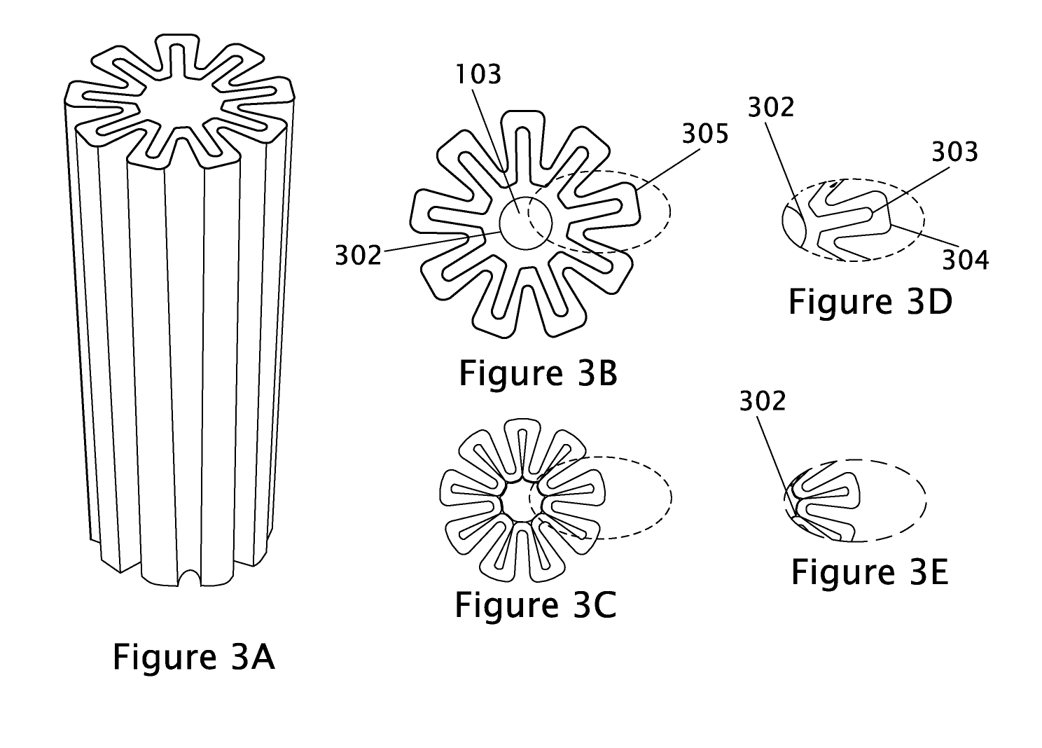

FIG. 3: The star-shaped collet (202) is shown in greater detail. FIG. 3A indicates a perspective view of the collet (202) and a radial cross section with cable (103) inserted is indicated in FIGS. 3B-3E. The inside (303) and outside (304) surfaces of the wall of each collet finger (305) are indicated as well as the contacting surface (302) of the cable (103).

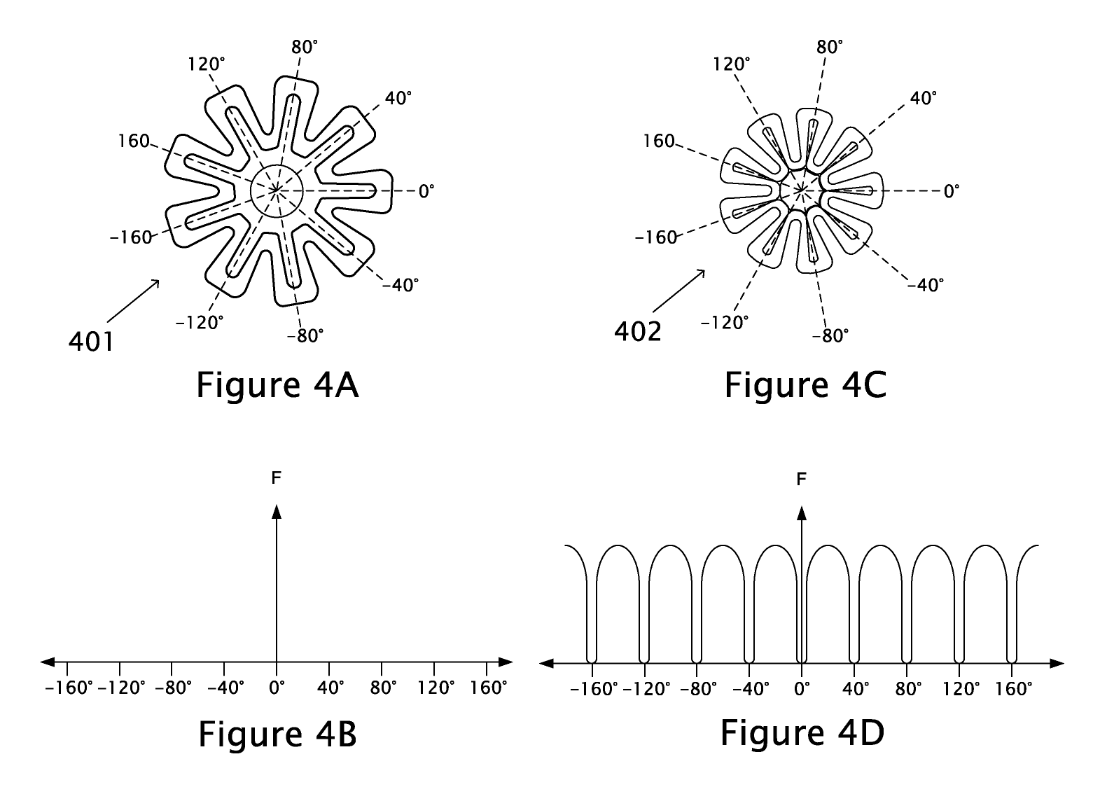

FIG. 4: A radial cross section of the star-shaped collet (202) with inserted surgical cable (103) is shown in an uncompressed state (401) in FIG. 4A. Its compressed state (402) is indicated in FIG. 4C. FIGS. 4B and 4D notionally indicate the degree of force area (404) between the collet (202) and surgical cable (103) in FIGS. 4A and 4B, respectively.

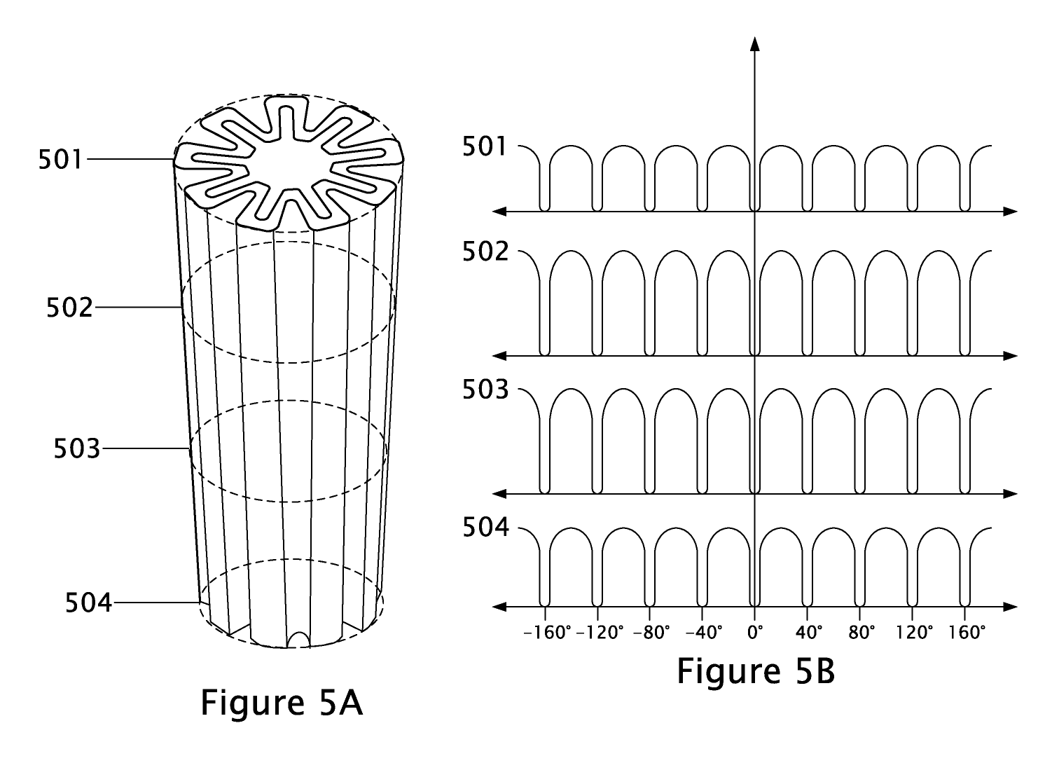

FIG. 5: FIGS. 5A-5B notionally indicate the degree of force area (404) for several positions (501)-(504) along the collet (202) length.

FIG. 6: Prior art cable (10) showing inner core (14) and outer braid (15).

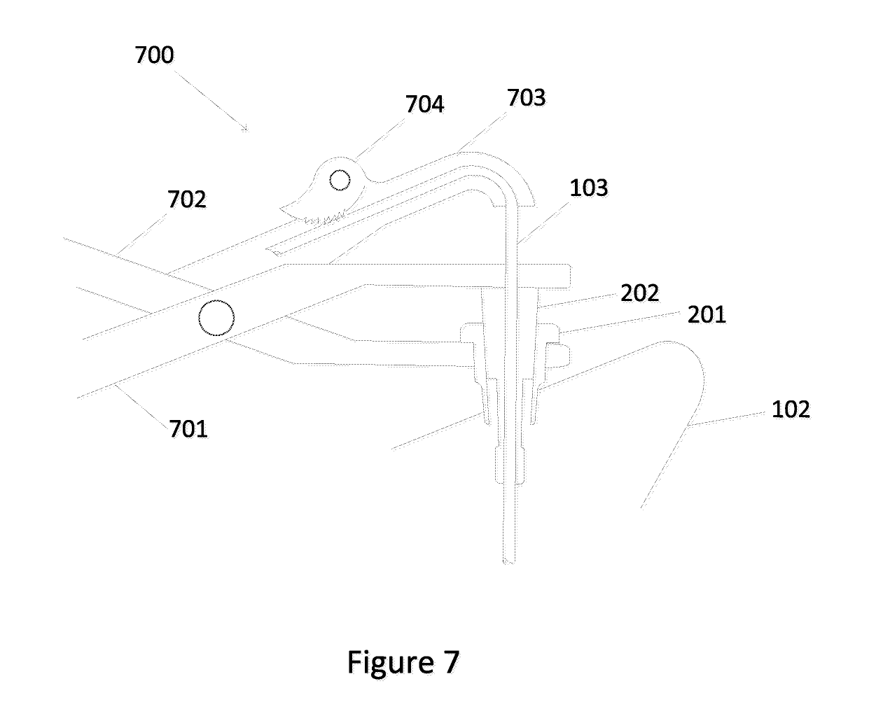

FIG. 7: Tool for installation of surgical cartridge (101) and surgical cable (103) inserted therein.

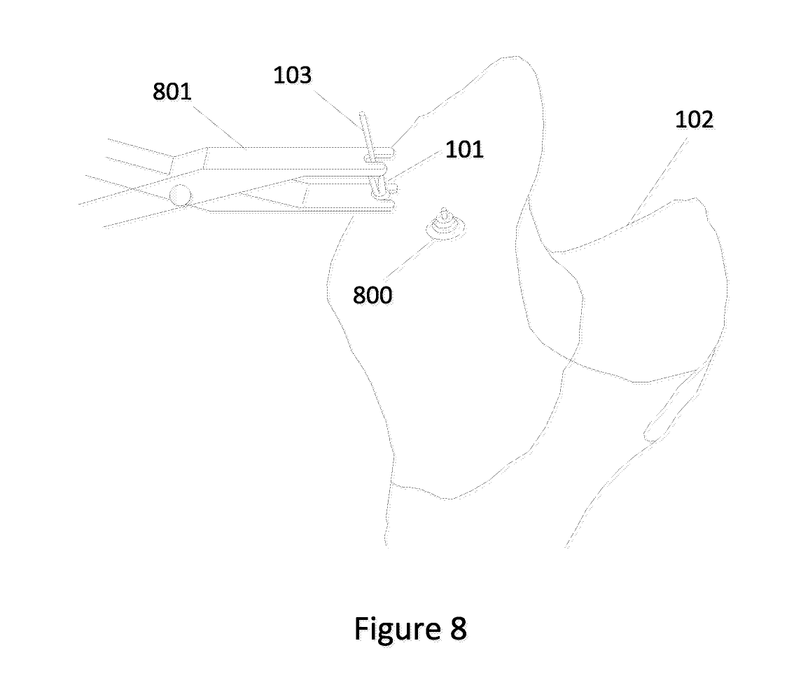

FIG. 8: Installation tool (700) detailing features and use of upper and lower forked members (701), (702).

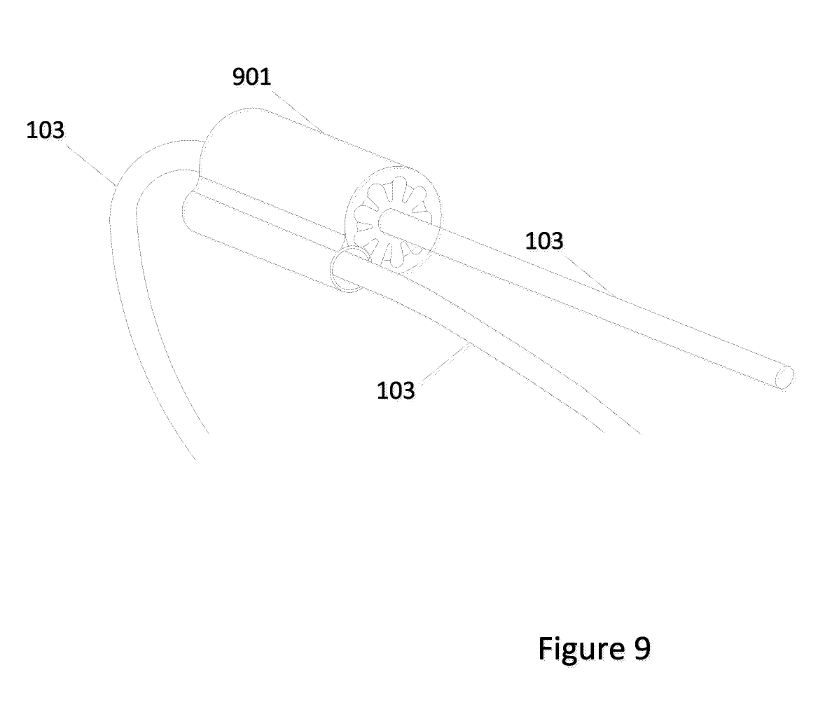

FIG. 9: Cerclage cartridge (901) with surgical cable (103) installed therein.

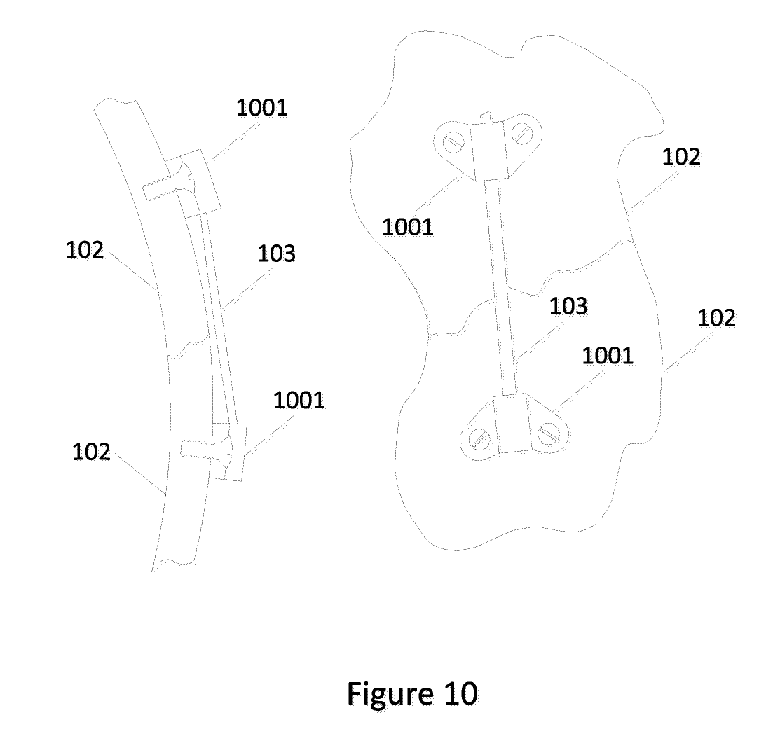

FIG. 10: Front and side views showing fractured flat bones (102) held in compression by a surgical cable (103). Each end of the cable is secured by a tabbed cartridge (1001) screwed into the flat bone segments.

FIG. 1 illustrates the use of the surgical cartridge. A surgical cable (103) has been threaded though bone fragments (102) as well as the central void (203) of the surgical cartridge (101). A tool (not shown) is then used to draw up, apply the required tension to the surgical cable (103), and seat the surgical cartridge (101) into the outer bone aperture (105). An optional washer (104) may be interspersed between the surgical cartridge (101) and the outer bone aperture (105).

FIG. 2 shows the surgical cartridge (101) in greater detail. The retaining collar (201) holds the star-shaped collet (202) and the central void (203) accommodates the surgical cable (103).

The star-shaped collet (202) is detailed in FIGS. 3A-3E. The perspective view shown in FIG. 3A illustrates the lengthwise features on the exterior of the collet (202). FIGS. 3B-3E illustrate varying stages of deployment of the collet onto the inserted cable (103). FIG. 3B along with the enhanced insert of FIG. 3D, indicate the offset position of the inside surface of the collet wall (303) with respect to the contacting surface (302) of the cable (103) for one collet finger (305). Here, the collet (202) is uncompressed, thus the cable slides freely along the central void (203) of the collet (202). By contrast, FIG. 3C along with the enhanced insert of FIG. 3E, indicate similarly with collet (202) in a compressed (deployed) state. Here, the inside surface of the collet wall (303) is pressed tightly against the contacting surface (302) of the cable (103), thereby preventing movement of the cable (103) along the central void (203) of the collet (202). The ability of the collet (202) to be compressed in this manner while maintaining physical integrity is determined by both the collet (202) material as well as the thickness of the collet wall, the approximate distance between its inner (302) and outer (303) surfaces. Both factors are important design parameters.

A radial cross section of the star-shaped collet (202) with inserted surgical cable (103) is again shown in an uncompressed state in FIGS. 4A-4B, with the corresponding illustrations for the compressed state shown in FIGS. 4C-4D. FIGS. 4A and 4C are simply re-rendered versions of FIGS. 3B and 3C with angular reference axis superimposed thereon. FIGS. 4B and 4D show a notional depiction of applied pressure, P, versus circumferential angle for FIGS. 4A and 4C, respectively. In the uncompressed state, FIGS. 4A-4B, the pressure exerted by the collet (202) on the cable (103) is zero for all angles since there is no contact between the inside surface of the collet wall (303) and the contacting surface (302) of the cable (103). By contrast, the notional depiction of applied pressure, P, versus angle shown in FIG. 4D for the compressed state depicted in FIG. 4C, indicates a regular non-zero behavior. For example, the pressure is minimal at 0°, ±40°, ±80°, ±120°, and ±160° because, at these points the inside surface of the collet wall (303) folds away from the contacting surface (302) of the cable (103). Alternatively, the pressure is maximal at 20°, ±60°, ±100°, ±140°, and 180° since these are points of maximal compression.

Of course, the detailed behavior of a working collet (202) is determined primarily by the shape and number of collet fingers (305). However, an optimal number of fingers is a design parameter that must be determined in balance with the parametrical design consideration of wall thickness. As earlier stated, a nine finger design appears to function well.

FIG. 5 shows a notional depiction of applied pressure, P, versus angle for various locations (501)-(504) along the length of the collet (202). Although a slight variation in maximal pressure is notionally indicated in the figure, this deviation should be made as minimal as possible.

If such concerns have been adequately addressed, the total force exerted on the cable (103) should be spread over as large an area as possible so that the applied pressure for any given unit surface area is not unduly high, thereby risking tearing and shearing of the delicate fibers covering the cable's outer surface. Consider a length of the cable (10) of FIG. 6. The braided fibers (15) wind around the core (14) in a helical fashion. Some fibers wind to the left while others wind to the right; both groups knit together to make up the braid. The relatively broad fingers of the collet capture this braided arrangement of crossed helical fibers, consistently engaging them in a balanced manner about the core circumference as the cable shrinks under tension.

FIGS. 7-8 illustrate an exemplary installation tool (700) for deployment of the surgical cartridge (101) and with a surgical cable (103) inserted therein. The installation tool (700) is of scissor-like design, having an upper forked member (701) and a lower forked member (702) for gripping the lower ledge of the retaining collar (201) and the upper surface of an inserted collet (202). A auxiliary lever, the cable gripping member (703), provides a positioning conduit through which the surgical cable (103) is threaded and tightened to the required level of tension. The tension is maintained via a tension retaining member (704). The upper forked member (701) and lower forked member (702) not only allow the cartridge assembly to be properly positioned, but also facilitate sliding the collet (202) into the retaining collar (201), thereby providing a gently increasing and uniform grip on a tightened surgical cable (103).

Installation of the surgical cartridge (101) with surgical cable (103) threaded therein occurs as follows:
1. The internally attached surgical cable (103) is threaded through the surgical cartridge (101) assembly,
2. The surgical cartridge (101) is pressed against the bone as the surgical cable (103) is pulled taut,
3. The upper forked member (701) and lower forked member (702) are squeezed together, urging the collet (202) into the throat of retaining collar (201), thereby closing in on the surgical cable (103) inserted therein.
4. The tension on the surgical cable (103) is released,
5. The upper and lower fork members (701), (702) are slid away from the surgical cartridge (101) assembly, and
6. The extraneous cable length is cut.

A cerclage cartridge (901) with an integrated attachment site is shown in FIG. 9. A first end of the surgical cable (103) is crimped into the attachment site by conventional means since, in its untensioned state, the surgical cable (103) is relatively soft and bulky. The free end is then positioned as required and the cable pulled taught. In this tensioned state, the diameter of the surgical cable (103) shrinks and the cable as a whole becomes taut and slippery, requiring the gentle radial compression afforded by the cartridge to effectively secure the remaining end. Consequently, the cerclage cable (901) is particularly well suited to address attachment of the surgical cable (103) in either circumstance.

FIG. 10 shows front and side views of a fractured flat bone (102) held in compression by a surgical cable (103). Tabbed cartridges (1001) screwed into the flat bone segments secure each end of the surgical cable (103).

Alternate embodiments envisioned but not shown include one or more surgical cartridges (101) integrated with other attachment devices such as a bone plate. Indeed, reconstruction techniques may include one or several surgical cartridges securing a network of surgical cables (103) and other attachment devices. In addition, the surgical cartridge (103) can be used to secure other medical tethers, such as (for instance), spider silk.

Recalling the objectives stated in the introductory section of this disclosure, the present invention provides a bio-medically compatible gripping device capable of radial collapse in accordance with the shrinkage of nylon or other polymeric material cored surgical cable undergoing tension while maintaining a firm grip throughout the process. Moreover, it provides a gripping device capable of maintaining a grip on the outer surface of a slippery delicate cable, the grip being approximately uniform along both the length and circumference of the cable. It also provides a gripping device capable of maintaining a grip, yet not damage, a delicate cable under high tension for a period of time adequate for as the healing process to occur.

While several illustrative embodiments of the invention have been shown and described, numerous variations and alternate embodiments will occur to those skilled in the art, and can be made without departing from the spirit and scope of the invention as defined in the appended claims.

1) A bio-compatible high tension suture anchor capable of providing a consistently uniform pressure of 2500-3500 psi over an area measuring less than 0.05 square inches.

2) A bio-compatible high tension suture anchor as in claim 1 further comprising:
a) A truncated, hollow, conical cylinder having a length, a radius, and a cylindrical wall, wherein said cylindrical wall is comprised of a regular series of ridges and valleys parallel to said length of said conical cylinder, and
b) A retaining collar operable for progressively compressing the radius of said conical cylinder upon insertion of said conical cylinder within said retaining collar.

3) A bio-compatible high tension suture anchor as in claim 2 wherein said regular series of ridges and valleys define a radial arrangement of compressive fingers, said fingers being operable for radially uniform compression of a cable inserted within the hollow space of said conical cylinder.

4) A bio-compatible high tension suture anchor as in claim 3 wherein said radially uniform compression is approximately invariant over said length of said conical cylinder.

5) A bio-compatible high tension suture anchor as in claim 4, further including a crimping tube integral with said suture anchor and parallel to said length of said conical cylinder.

6) A bio-compatible high tension suture anchor as in claim 4, further including a pair of opposing attachment tabs integral with said suture anchor and perpendicular to said length of said conical cylinder.

7) A bio-compatible high tension suture anchor as in claim 4 wherein said radially uniform compression is consistent over cable diameter shrinkages of up to 15%.

8) A bio-compatible high tension suture anchor as in claim 7 having 7-11 radial fingers.

9) A bio-compatible high tension suture anchor as in claim 8 wherein said radial fingers have a wall thickness of 0.012-0.012 inches.

10) A bio-compatible high tension suture anchor as in claim 9 wherein said high tension suture is made of a metallic alloy.

11) A bio-compatible high tension suture anchor as in claim 10 wherein said metallic alloy is titanium.

12) A bio-compatible high tension suture anchor as in claim 11, further including a crimping tube integral with said suture anchor and parallel to said length of said conical cylinder.

13) A bio-compatible high tension suture anchor as in claim 11, further including a pair of opposing attachment tabs integral with said suture anchor and perpendicular to said length of said conical cylinder.

14) An exemplary installation tool for deployment of a high tension suture anchor, said tool comprising;
a) An upper forked member and a lower forked member, wherein said upper forked member and said lower forked member are configured in a scissor-like fashion, wherein said lower forked member is operable for securing and positioning said high tension suture anchor,
b) An auxiliary lever operable for gripping a surgical cable threaded through the high tension suture anchor, wherein said auxiliary lever comprises a positioning conduit operable for positioning and tightening a surgical cable to the desired level of tension, and
c) a tension retaining member, wherein said tension retaining member is operable for maintaining the desired level of tension on said surgical cable throughout the deployment process.

15) A method of deploying a surgical cable within a fractured bone, said method comprising the steps of:
a) Presenting a high tension suture anchor as in claim 4,
b) Presenting an installation tool as in claim 14,
c) Positioning the anchor within the tool, thereby forming a tooled anchor assembly,
d) Threading a surgical cable through said tooled anchor assembly,
e) Pressing said anchor assembly through a surgically drilled bone aperture while pulling said surgical cable taut,
f) Squeezing the upper and lower forked members of said installation tool together, while urging the conical cylinder of said anchor into the throat of its retaining collar, thereby gripping and securing the inserted surgical cable,
g) Releasing the tension on said surgical cable,
h) Sliding said upper and lower fork members away from said anchor assembly, and
i) Cutting the extraneous length of said surgical cable.

16) A method of employing cerclage to an assembly of fractured bones using a surgical cable, said method comprising the steps of:
a) Presenting a high tension suture anchor as in claim 5,
b) Presenting an installation tool as in claim 14,
c) Positioning the anchor within the tool, thereby forming a tooled anchor assembly,
d) Crimping one end of said surgical cable in said crimping tube,
e) Encircling said assembly of fractured bones with the free end of said surgical cable,
f) Threading said free end of said surgical cable through said tooled anchor assembly,
g) Squeezing the upper and lower forked members of said installation tool together, while urging the conical cylinder of said anchor into the throat of its retaining collar, thereby gripping and securing the inserted surgical cable,
h) Releasing the tension on said surgical cable,
i) Sliding said upper and lower fork members away from said anchor assembly, and
j) Cutting the extraneous length of said surgical cable.

17) A method of deploying a surgical cable to the surface of a fractured bone, said method comprising the steps of:
a) Presenting a high tension suture anchor as in claim 6,
b) Presenting an installation tool as in claim 14,
c) Positioning the anchor within the tool, thereby forming a tooled anchor assembly,
d) Threading a surgical cable through said tooled anchor assembly,
e) Pressing said anchor assembly against the surface of said fractured bone such that said opposing tabs lie flat against the bone,
f) Screwing said anchor assembly to said fractured bone using said opposing tabs,
g) Pulling said surgical cable taut,
h) Squeezing the upper and lower forked members of said installation tool together, while urging the conical cylinder of said anchor into the throat of its retaining collar, thereby gripping and securing the inserted surgical cable,
i) Releasing the tension on said surgical cable,
j) Sliding said upper and lower fork members away from said anchor assembly, and
k) Cutting the extraneous length of said surgical cable.
